(19) United States
(12) Patent Application Publication
(10) Pub. No.: US 2009/0118359 A1
(43) Pub. Date: May 7, 2009

(54) Food product

(75) Inventors: Leo Brunia, ATVlaardingen (NL); Robertus Martinus M. Diks, ATVlaardingen (NL)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco, Inc., d/b/a Unilever

(21) Appl. No.: 12/290,840

(22) Filed: Nov. 4, 2008

(30) Foreign Application Priority Data
Nov. 6, 2007 (EP) EP07120071

(51) Int. Cl. A61K 31/352; A61P 9/10

(52) U.S. Cl. 514/456

(57) ABSTRACT

A method for the treatment of a bioflavonoid composition, comprising the contacting of the bioflavonoid composition with ethanol of at least 90% purity at a temperature of from 10 to 80° C. for a period of at least 0.5 hour, followed by separation of the ethanol and the bioflavonoid, wherein the contacting comprises the solubilisation of the bioflavonoid in the ethanol and whereby the separation involves the recrystallisation of the bioflavonoid and whereby the contacting takes place in the presence of active carbon in an amount of 1-10 wt % based on the bioflavonoid.

### FIELD OF THE INVENTION

The invention relates to a food product comprising one or more bioflavonoids. The invention also relates to a method to improve the quality of crude bioflavonoid compositions.

### BACKGROUND OF THE INVENTION

Food products have previously been suggested as vehicles for incorporation of healthy ingredients, such as blood-cholesterol lowering ingredients e.g. like sterols, blood-pressure lowering ingredients, for example peptides, long-chain poly unsaturated fatty acids (LC-PUFA) from fish-oil, poly phenols, anti-oxidants and many more.

It has been suggested in WO 00/15174 to incorporate bioflavonoids in foods and beverage compositions.

It is an object of the invention to formulate and manufacture food products which comprise bioflavonoid ingredients at a level suitable to alter the lipid profile in the blood of consumers, in particular to reduce the cholesterol level in the blood of consumers.

The incorporation of bioflavonoids, particularly citrus bioflavonoids, in food compositions however encounters a number of problems. For example often commercially available citrus bioflavonoid compositions cannot readily be used in food products because these provide an undesired brown colour and/or an undesired off-flavour to the product. Furthermore an analysis of crude citrus bioflavonoid extract has shown the occasional presence of undesired substances such as for example polycyclic aromatics or dioxins.

Additionally the incorporation of such bioflavonoids in food products often is difficult to achieve because the bioflavonoids often seem to have a negative influence on the product characteristics, for example a negative impact on taste or negative impact on mouthfeel. Furthermore applicants have encountered problems to stably and or homogeneously incorporate bioflavonoid into food structures because they lead to more complicated manufacturing processes and to a negative impact on product stability.

A first object of the invention is to provide a process to treat a bioflavonoid composition, particularly a citrus bioflavonoid composition, which can overcome one or more of the above problems. Another object of the invention is to define an easily measurable parameter, which can serve to distinguish suitable bioflavonoid compositions from less suitable bioflavonoid compositions. Another object of the invention is to provide food products which comprise the bioflavonoid compositions obtained by the method of the invention or otherwise satisfying the above described parameter.

Another object of the invention is to provide a food product which is stable under storage at ambient and optionally higher temperatures. Further food products with good organoleptic and/or visual properties are envisioned with the current invention.

EP1576893 describes the extraction of isoflavones from soybean by using a 70% by volume aqueous ethanol. As illustrated in the examples (see below) such extraction results in relatively high levels of contamination and is therefore less suitable.

US2003/108591 describes the combined use of sterol esters and isoflavones in foods.

Cheminfo XP 002478773 describes the compound benzo(a)pyrene and confirms its possible carcinogenic nature.

U.S. Pat. No. 2,630,432 describes the extraction of naringin from grapefruit molasses using 95% ethanol.

U.S. Pat. No. 6,528,099 describes the extraction of hesperidin from orange peel by column extraction with 96% ethanol.

Surprisingly it has been found that some or all of the above problems can be resolved by a careful pre-treatment of bioflavonoid compositions. In particular it has been found that specific extraction conditions provide a bioflavonoid composition of improved quality which is suitable for the incorporation of foods such as spreads and drinks.

In a first aspect the invention relates to a method for the treatment of a bioflavonoid composition, comprising the contacting of the bioflavonoid composition with ethanol of at least 90% purity at a temperature of from 10 to 80° C. for a period of at least 0.5 hour, followed by separation of the ethanol and the bioflavonoid, wherein the contacting comprises the solubilisation of the bioflavonoid in the ethanol and whereby the separation involves the recrystallisation of the bioflavonoid and whereby the contacting takes place in the presence of active carbon in an amount of 1-10 wt % based on the bioflavonoid. Preferably the bioflavonoid composition is a citrus bioflavonoid composition, for example obtainable by extracting citrus peels.

In a first advantageous embodiment of the invention the contacting of the bioflavonoids and the ethanol takes place at relatively low temperatures e.g. 10-25° C. During this contacting the bioflavonoids will generally remain unsolubilized. Afterwards the extracted bioflavonoids can be separated from the ethanol by convenient ways such as for example evaporation, filtration or centrifugation.

Typical contacting times for this embodiment are from 1-10 hours, for example 2-5 hours.

In a second advantageous embodiment of the invention the contacting of the bioflavonoids and the ethanol takes place at an elevated temperature, say from 30 to 80° C. to allow the solubilization of the bioflavonoid in the ethanol. Afterwards the solubilized bioflavonoids can be separated from the ethanol by convenient ways such as for example by cooling the ethanol to say 0 to 25° C. to effect the recrystallisation of the bioflavonoid from the ethanol.

Advantageously the contacting of the ethanol with the bioflavonoids before recrystallisation can be in the presence of active carbon, for example at an amount of 1-10 wt % based on the bioflovonoids. Advantageously the active carbon can be separated by conventional means e.g. filtration, where after the cooling to allow recrystallization can take place.

Preferred ways of carrying out the method of the invention are described in the Examples.

In a further aspect of the invention it has been found that the benzo(a)pyene content of the bioflavonoids can advantageously be used to determine whether the treated bioflavonoid composition can suitably be used for the incorporation in food product whereby negative qualities such as a brown colour and severe off-taste can be minimised. In particular it has been found that a benzo(a)pyrene (BAP) content of less than 2 ppm is an excellent marker of suitable quality for the purified flavonoid composition. Therefore the present invention relates to a bioflavonoid composition comprising less than 2 ppm (parts per million) of benzo(a)pyrene, more preferred from 0.1 to 2 ppm, most preferred from 0.1 to 1 ppm. Also preferably suitable bioflavonoid compositions are distinct over crude untreated bioflavonoid compositions in that the colour is not darkish brown but rather light brown to (light) yellow.

It has also been found that the purified flavonoid compositions of the invention are especially suitable for the incorporation into specific food products, in particular spreads and drinks.

Accordingly in a third aspect the present invention relates to a vegetable oil based spreads in the form of a water in oil emulsion, said emulsion comprising:
(a) from 20 to 85 wt % of vegetable fat;
(b) from 0.01 to 5 wt % of bioflavonoids,
(c) wherein the content of benzo(a) pyrene relative to the bioflavonoids is less than 2 ppm.

Accordingly in a fourth embodiment the invention relates to a drink, especially a dairy based drink, wherein the drink comprises
(a) from 10 to 99 wt % of a liquid protein base, for example a dairy base such as cow milk or yoghurt or a vegetable protein base such as soy milk; and
(b) from 0.01 to 5 wt % of bioflavonoids; and
(c) wherein the content of benzo(a) pyrene relative to the bioflavonoids is less than 2 ppm.

### DETAILED DESCRIPTION OF THE INVENTION

Bioflavonoids are natural components obtainable from, for example, fruits and vegetables. For the purpose of the invention bioflavonoids are preferably those bioflavonoids which can be obtained from citrus fruits for example by extraction of citrus peels. These bioflavonoids are for the purpose of the invention referred to as citrus bioflavonoids. Especially preferably the bioflavonoids for use in the invention are for more than 50 wt %, for example 70-100 wt %, such as 90-100 wt % polymethoxylated flavones.

Crude citrus bioflavonoid materials are commercially available for example via KGK or One Source Global under the tradename Sytrinol.

Crude citrus bioflavonoid compositions often contain a mixture of flavonoid materials, such as for example one or more of hesperedin, naringin, tangeretin, nobiletin etc. Crude citrus bioflavonoid compositions generally have a brown or sometime green-grey colour. Examples of crude citrus bioflavonoid materials are for example Citrus Nobilis extract ex KGK and Citrus Aurantium from Fenchem, Advantra Z from Nutritech.

In the method according to the invention the (generally brown) (citrus) bioflavonoid composition is contacted with ethanol of at least 90% purity at a temperature of from 10 to 80 C for a period of at least 0.5 hour. Preferably the weight ratio of (citrus) bioflavonoids to ethanol will be from 1:1 to 1:10, more preferably from 1:2 to 1:5. Typically the temperature could for example be between 20 and 70 C. At this temperature the contact time between the ethanol and the bioflavonoid is advantageously from 0.5 to 5 hours, fore example 1-3 hours, after that either the temperature can be reduced (e.g. to allow the recrystallization or the (citrus) bioflavonoid can be removed e.g. by filtration.

Spreads of the inventions are vegetable oil based spreads of the water in oil type. Such spreads are for example used as low or full-fat margarine type product, for example for the flavouring of food products or the spreading on for example sandwiches and toasts. Vegetable oil based spreads may sometimes also be used for baking or frying purposes. In addition of water and vegetable oil spreads of the invention may comprise various ingredients and flavouring ingredients for example as described here below.

The spreads of the invention may optionally comprise thickeners. For stability reasons it may be useful to include thickeners in the emulsion, for example in low fat spreads containing 20 to 40 wt % of fats, often improve by addition of thickeners. Whether or not a thickener should be added and in what amount depends on factors as stability and application and may be determined by the skilled person.

Suitable thickeners may be any known thickener and are preferably selected from the group comprising gums, like xanthan, guar, and locust bean, carrageenan, polysaccharides, alginate, pectin, starch, and gelatin. The level of thickener in compositions of the invention, preferably is from 0.1 to 5 wt %.

In preferred spread products according to the invention, the aqueous phase comprises a fully gelatinised starch selected from any of the main starch groups: wheat, potato, rice, maize, waxy rice or waxy maize.

The amount of starch in the food product according to the invention depends somewhat on the type of chosen starch and is preferably from 0.2 to 5 wt %, more preferred from 0.7 to 3 wt %, most preferred from 1 to 2 wt %.

To ensure homogeneous distribution of the aqueous phase in the continuous fat phase, the droplet size distribution D_{3,3} of the dispersed aqueous phase is preferably less than 8 μm, more preferably from 4 to 20 μm, more preferred even lower than 10 μm. It will be appreciated that the droplet size can be controlled by adjusting the processing conditions in the unit operations: e.g. higher rotational speed in a scraped surface heat exchanger will produce correspondingly smaller water droplet size distributions.

The spreads according to the invention comprise from 20 to 85 wt % of a vegetable fat, preferably from 30 to 80 wt %, most preferably from 35 to 60 wt %.

The fat can be a single fat or a combination of vegetable fats. The fat or combination of fats is preferably selected such that the solid fat content is below 6% at 35° C., preferably below 5% at 35° C., more preferred below 4% at 35° C., most preferred from 2 to 4% at 35° C. Optionally relatively small amounts of non-vegetable fats, for example animal fats such as butter or marine oils, for example at levels of 0.1 to 25 wt %, more preferred 0.1 to 5 wt % may advantageously be present in the spreads of the invention.

Suitable vegetable fats can for example be selected from the group comprising bean oil, sunflower oil, palm kernel oil, coconut oil, palm oil, rapeseed oil, cotton seed oil, maize oil, or their fractions, or a combination thereof. Inter esterified fat blends of these fats or optionally with other fats are also encompassed in the invention.

Advantageously, marine oils such as fish oil and or algae oil may be added for the addition of omega-3 and omega-6 fatty acids.

Spreads according to the invention comprise from 0.01 to 5 wt % of the bioflavonoids preferably selected from Tangeretin and Nobiletin or mixtures thereof.

Preferably the total level of bioflavonoids, particularly citrus flavonoids is from 0.5 to 4 wt %, most preferred from 1 to 3 wt %.

Preferably the (citrus) bioflavonoids are largely composed of tangeretin or nobiletin or mixtures thereof in the preferred total amounts for the combination of these ingredients as specified above. Especially preferred is the use of nobiletin at a level of from 0.5 to 1.5 wt %.

Preferably spreads according to the invention comprise an emulsifier such as polyglycerol polyricinoleate, distilled monoglycerides, citric acid esters of monoglycerides, di-acetyl acetic acid esters of monoglycerides, lactic acid esters of monoglyceride, mono-diglycerides, polyglycerol esters of fatty acids or sorbitan esters of fatty acids.

The most preferred emulsifiers are polyglycerol polyricinoleate and monoglycerides. Even more preferred are combinations of a monoglyceride comprising a saturated fatty acid residue and a monoglyceride comprising an unsaturated fatty acid residue.

The amount of emulsifier depends on the type and effectiveness of the emulsifier selected and can be determined by the person skilled in the art. As a general guidance the amount of emulsifier is preferably from 0.05 to 1.5 wt %, more preferred from 0.1 to 0.7 wt %, most preferred from 0.15 to 0.5 wt %.

The pH of the aqueous phase of the spread can be set to the desired value, among others to influence acidic or basic taste impression and to influence microbial stability. Preferably the pH of the aqueous phase in food products according to the invention is from 4.3 to 5.5.

Optionally some protein may be added to the spread according to the invention. Protein may be added to beneficially influence the taste, flavour and nutritional value of the food product and also may be added to increase browning of food stuff when the current composition is used as a medium for shallow frying. Generally the level of protein may for example be from 0.1 to 10 wt %.

The spreads according to the invention optionally contain other ingredients such as preservatives, vitamins, taste and flavour components, colorants such as beta-carotene, anti-oxidants.

The food product according to the invention can be prepared by any suitable process to prepare such products.

For example the preparation of a food product according to the invention comprises the preparation of an aqueous phase prepared comprising starch and water and other water soluble ingredients, which aqueous phase is heated to a temperature from 60 to 95° C. for at least 15 minutes to gelatinise the starch such that at least 50% is gelatinised, and subsequently cooled to a temperature of from 50 to 70° C., and separately a fat phase is prepared comprising fat phase ingredients at a temperature of around 60° C. and in a further step the aqueous phase and the fat phase are mixed at a temperature around 60° C.

Spreads of the invention can be used to lower the level of serum cholesterol. Preferably this use involves the use of 5-50 g, more preferred 10-25 g of spread per day, for example in the form of 1-10 slices of bread, for example 1-5 slices each slice comprising on average 5 g of the spread.

Drinks of the invention can be manufactured by any suitable method, for example by simply mixing the protein base, for example in the form of milk, yoghurt or soymilk with the remainder of the ingredients such as fruit juice, sweeteners, thickeners, citrus bioflavonoids etc. By way of example a possible range of ingredients for use in drinks is for example 10-99 wt % of liquid protein base, preferably (cow) milk, soy milk or yoghurt, 0.01 to 5 wwt % of bioflavonoids, preferably these levels apply to citrus flavonoids, particularly polymethoxylated flavones, even more particular the composition comprises 0 nobiletin at a level of 0.5 to 1.5 wt %, 0.01 to 20 wt % of sweetening ingredients e.g. sugar or artificial sweeteners and 0 to 35 wt % of fruit juice or fruit pulp. For yoghurt based drinks the pH generally will be slightly acidic e.g. from 4 to 5, for milk based drinks generally a neutral to slightly elevated pH is commonly used.

Drinks of the invention advantageously are heat treated e.g. pasteurized or sterilized and packed in closed containers of 50 to 2000 mls, for example 50 to 200 ml.

### Experimental:

### Storage Stability Test

Food product was stored in a plastic container at 10, 20, 30, 35 and 40° C. for up to 26 weeks. After storage the amount of phase separation was determined by visual examination of the product surface. Storage stable products show a phase separation of less than 5 wt % upon storage at 35° C. for at least 10 weeks, preferably at least 26 weeks. Preferably the phase separation is less than 5 wt % upon storage at 40° C.

### Method to Determine D_{3,3}

The water droplet size was measured using a well known low resolution NMR measurement method. Reference is made to Alderliesten, M.; Part. Part. Syst. Charact. 8 (1991), 237-241.

### Method to Determine Solid Fat Content

The solid fat content can be measured by a suitable analytical method such as NMR. The method used is low resolution NMR with Bruker Minispec apparatus. Reference is made to the Bruker minispec application notes 4,5 and 6.

The percentage of solid fat determined by the low resolution NMR technique is defined as the ratio of the response obtained from the hydrogen nuclei in the solid phase and the response arising from all the hydrogen nuclei in the sample. The product of this ratio and one hundred is termed the low resolution NMR solids percent. No correction is made for variations in the proton density between solid and liquid phase. The NMR solids percent for a sample measured at t ° C. was given the symbol Nₜ.

Suitable instruments adapted to determine the solids fat content are the Bruker Minispecs p₂₀i™, pc20™, pc120™, pc120™, NMS120™ and MQ₂₀™.

Stabilization and tempering procedure was as follows:
melt fat at 80° C.
5 minutes at 60° C.
60 minutes at 0° C.
30-35 minutes at each chosen measuring temperature.

### EXAMPLE I

### Extraction with 96% Ethanol

100 gram crude brown bioflavonoid material (citrus nobilis) ex KGK. Batch code (LBR200701-030) composed of 94% citrus bioflavonoids (1% Sinensetin, 75% Nobiletin,3% Desm. Nobiletin, 14% Tangeretin, 1% other flavones) was mixed with 400 gram ethanol 96% at ambient temperature in a conical flask with a magnetic stirrer for 1 hour. The mixture was filtered over a 3μ filter, the residue was rinsed with 50 gram cold (<0° C.) ethanol 96%. The residue was dried for about 14 hours at 50° C. and weighted. Both crude and purified material were analysed on PAH's.

### EXAMPLE II

### Treatment Re-Crystallisaton of the Bioflavonoids

51.5 gram crude KGK material of Example I was mixed with 200 gram 96% ethanol at 50° C. until all citrus bioflavonoids were dissolved in ethanol. The mixture contains 20% KGK material.

0.75% (=1.8 gram) Norit SA4 PAH FF active carbon (FPK200303-086) was added and the mixture was stirred with a magnetic stirrer for 1 hour at 50° C. After this the active carbon was removed by filtration over a 2μ filter. The filtrate was stored for 3 days at ambient temperature (˜20° C.) in order to let bioflavonoids re-crystallize. The mixture was filtered over a 2μ filter and the residue was dried for about 14 h at 50° C. and analysed on PAH's.

### Results

The following results were obtained:

The BAP content of the crude bioflavonoid material was 170 ppm, the dioxin content of the crude material was 0.70 ppm.

The extracted bioflavonoid of example I had a BAP content of 4.1 ppm and a dioxin content of 0.036 ppm.

The recrystallised bioflavonoid of Example II had a BAP content of 0.5 ppm and a dioxin content below the detectable level.

Both extracted (ExI) and recrystallised bioflavonoid (Ex II) had an significantly improved colour and taste as compared to the crude citrus flavonoid material. Both materials were no longer brown but light yellow of colour.

### EXAMPLE III

A vegetable oil based spread was prepared with the following composition (parts by weight)

| Ingredient | Weight parts |
|---|---|
| PLANT STEROLESTERS¹⁾ | 12.6 |
| Fat 1²⁾ | 10.2 |
| Fat 2³⁾ | 8.4 |
| Hardstock1⁴⁾ | 8.2 |
| Hardstock 2⁵⁾ | 2.1 |
| Rapeseeed oil | 0.7 |
| Recrystalised bioflavonoid (example II) | 1 |
| SYNTHETIC B-CAROTENE | 0.2 |
| DIMODAN HP emulsifier | 0.2 |
| Lecithin SUNLEC Z | 0.1 |
| DL-A-TOCOPHEROL ACETATE | 0.02 |
| FLAVOUR | 0.006 |
| TAPIOCA STARCH | 2.5 |
| SWEET WHEY POWDER | 0.4 |
| POTASSIUM SORBATE | 0.1 |
| Water | To 100 |

Notes:
¹⁾Sitosterol ester of sunflower oil.
²⁾Mixture of Linseed oil and Sunflower oil in weight ratio of 22:78
³⁾Mixture of Linseed oil and Linola oil in a weight ratio of 21:79
⁴⁾Interesterified blend of palm oil mid fraction (45 weight parts), sunflower oil (28 weight parts), palm kernel oil (15 weight parts) and linseed oil (12 weight parts).
⁵⁾Interesterified blend of palm oil mid fraction (65 weight parts) and palm kernel oil (35 weight parts)

The spread was prepared as follows: the fats, hardstocks, sterolesters, bioflavonoid, beta carotene, emulsifier, lecithin, tocopherol and flavour were premixed to form a fat phase.

The remaining ingredients were mixed to form a water-phase. The water phase was mixed with the fat phase to form a water in oil emulsion which was kept for 30 minutes at 62° C. and which was subsequently cooled and inverted into a water in oil emulsion by passing through 3 subsequent A units, followed by one C unit. The products were filled into tubs and stored at 5° C.

As a comparison the same spread was prepared using crude bioflavonoid materials. The spread with the recrystallized bioflavonoids had a markedly improved color (less brown) and taste (less bitter). Both spreads had a good mouthfeel and stability after storage.

1. A method for the treatment of a bioflavonoid composition, comprising the contacting of the bioflavonoid composition with ethanol of at least 90% purity at a temperature of from 10 to 80° C. for a period of at least 0.5 hour, followed by separation of the ethanol and the bioflavonoid, wherein the contacting comprises the solubilisation of the bioflavonoid in the ethanol and whereby the separation involves the recrystallisation of the bioflavonoid and
whereby the contacting takes place in the presence of active carbon in an amount of 1-10 wt % based on the bioflavonoid.

2. A method according to claim 1, wherein the bioflavonoid composition is a citrus bioflavonoid composition.

3. A method of claim 2, wherein the bioflavonoid composition has a brown colour.

4. A bioflavonoid composition comprising less than 2 ppm, preferably from 0.1 to 2 ppm of benzo(a)pyrene, more preferred from 0.1 to 1 ppm.

5. A bioflavonoid composition according to claim 4, being a citrus bioflavonoid composition.

6. A vegetable oil based spreads in the form of a water in oil emulsion, said emulsion comprising:
(a) from 20 to 85 wt % of vegetable fat;
(b) from 0.01 to 5 wt % of bioflavonoids,
(c) wherein the content of benzo(a) pyrene relative to the bioflavonoids is less than 2 ppm.

7. A drink, especially a dairy based drink, wherein the drink comprises:
(a) from 10 to 99 wt % of a liquid protein base, for example a dairy base such as cow milk or yoghurt or a vegetable protein base such as soy milk; and
(b) from 0.01 to 5 wt % of bioflavonoids; and wherein the content of benzo(a) pyrene relative to the bioflavonoids is less than 2 ppm.

8. A spread according to claim 6, comprising from 0.01 to 5 wt % of nobiletin.

9. A drink according to claim 7, comprising from 0.01 to 5 wt % of nobiletin
